(19) United States
(12) Patent Application Publication
(10) Pub. No.: US 2010/0234915 A1
(43) Pub. Date: Sep. 16, 2010

(54) Non-bioelectrical pressure-based sensing for temporary pacemakers

(76) Inventors: Michael B. Herlich, Voorhees, NJ (US); Steven D. DaTorre, Marlton, NJ (US); Michael J. Ceglia, Cherry Hill, NJ (US); S. Robert Miller, Bonita Springs, FL (US)

Correspondence Address: NORMAN E. LEHRER, P.C., 1205 NORTH KINGS HIGHWAY, CHERRY HILL, NJ, 08034, US

(21) Appl. No.: 12/661,044

(22) Filed: Mar. 9, 2010

Related U.S. Application Data
(60) Provisional application No. 61/210,072, filed on Mar. 11, 2009.

(51) Int. Cl. A61N 1/365

(52) U.S. Cl. 607/23; 607/17

(57) ABSTRACT

For temporary cardiac pacing, non-bioelectrical monitoring of intracardiac blood pressure variations is provided in the right ventricle. Unlike traditional bioelectric sensing, which is accomplished via the pacing leads, pressure based sensing can be independently accomplished from anywhere within the volume of the right ventricle, making it unnecessary to force a stiff pacing electrode tip into the myocardium to ensure quality sensing. Consequently, the distal pacing electrode can be designed with a more bulbous tip to significantly reduce the risk of myocardial perforation during implant. An inflatable bladder, employed initially to guide the catheter into the right ventricle, is subsequently employed as a fluid pressure sensor bulb to transmit intracardiac blood pressure variations to a fluid pressure transducer integral within the pacing controller.

### CROSS-REFERENCE TO RELATED APPLICATIONS

This application claims the benefit of U.S. Provisional Patent Application Ser. No. 61/210,072, filed Mar. 11, 2009.

### FIELD OF THE INVENTION

This invention describes a non-bioelectrical monitoring process for temporary pacemakers to sense (monitor) a patient's underlying cardiac rhythm using real time dynamic intracardiac blood pressure analysis.

### BACKGROUND OF THE INVENTION

A temporary pacemaker is a device utilized, either as a bridge to a permanent pacemaker or until the heart rate normalizes on its own, to trigger the heart to beat at a normal rate when the heart is beating too slowly. Occasionally these devices are utilized in situations where the possible development of bradycardia (slow heart beat) is a concern as, for example, during a percutaneous coronary intervention. In addition to a temporary pacemaker's ability to pace the heart, it must also have the ability to sense (monitor) the underlying cardiac rhythm so as not to interfere with the heart's intrinsic beats, which could lead to serious dysrhythmias. Current technology utilizes bioelectrical rhythm monitoring via the same electrical conducting leads that simultaneously serve to pace the heart. These intracardiac leads are on the distal tips of percutaneously inserted wires connected to an external pacing controller.

The ability to obtain quality cardiac sensing by temporary pacemakers is a true clinical concern for a variety of reasons. First, these devices are often placed in an emergency setting such as in resuscitation from sudden cardiac death or with a patient in bradycardic shock (hemodynamic collapse from low blood pressure caused by the slow heart rate) such that they are hastily placed in blind (no fluoroscopy) fashion. In these cases, the patients are more susceptible to potentially fatal myocardial (heart muscle) perforation given the rapidity of blind implant and the necessity to force the stiff electrode tip into the myocardium to ensure quality sensing (though unnecessary to secure consistent pacing). As well, a patient in shock is more susceptible to dysrhythmias that can be exacerbated by impaired sensing. Many clinicians concerned about this potential for serious temporary pacemaker implantation complications have chosen to use the less reliable transcutaneous pacing in these circumstances.

Given the need for this lifesaving technology, it would be desirable to have temporary pacemaker leads that could be implanted more safely in an emergency with reliable sensing in addition to consistent pacing. This would allow more universal application of this technology rather than the current over-reliance on transcutaneous technology in the emergency setting.

### OBJECTS AND ADVANTAGES

It is an object of this invention to describe a method for utilizing non-bioelectrical sensing for intracardiac monitoring in temporary transvenous pacing systems.

It is further an object of this invention to describe how non-bioelectrical sensing can be fulfilled by elements of the lead sets used to assist the implant surgeon in guiding, directing, and placing the lead sets.

It is further an object of this invention to provide examples of novel, inherently safer lead tip technology, enabled by incorporating non-bioelectrical sensing technology into the lead sets.

It is further an object of this invention to describe complementary functions in an external pacing controller to take advantage of the non-bioelectrical sensing technology.

Advantages of this invention include:
1. Reliable intracardiac sensing without risking myocardial perforation.
2. Safer lead tip technology.
3. Placement guiding element also fulfills non-bioelectrical sensor function.
4. Increased utilization of temporary transvenous pacing in the emergency setting (compared with the less reliable transcutaneous pacing) due to enhanced safety of implantation.

### SUMMARY OF THE INVENTION

This invention accomplishes its procedural objects by comparing real time cyclical changes in intracardiac blood pressure that result from the heart's pumping action. This information is collected and inserted into (unclaimed) algorithms that determine whether the heart should be paced or remain in monitoring mode if the heart is beating appropriately on its own.

In one embodiment, a dedicated fluid pressure transducer is placed on the lead set and attached to a connector that exits the proximal end of the lead set for connection to the external pacing controller along with the positive and negative connectors for the pacing leads.

In the preferred embodiment, an inflatable bladder, used initially to assist in placement of the lead set, is left inflated and in place to serve as an efficient and robust fluid pressure sensor bulb. Cardiac pressure variations and events are monitored by the pacing controller and used to determine when the heart requires pacing.

This invention encompasses options for pressure based sensing including but not limited to a fluid pressure transducer, a dedicated fluid pressure sensor bulb, and a dual purpose fluid pressure sensor bulb.

In the case of the dedicated fluid pressure sensor, the sensor is placed on a catheter attached to a connector that exits the proximal end of the lead set for connection to the external pacing controller along with connectors for the pacing leads. Cardiac pressure variations and events are monitored by the pacing controller and used to determine when the heart requires pacing.

In the preferred embodiment of this invention, a dual purpose fluid pressure sensor bulb comprises a balloon (bladder) guided pacing catheter, commonly used in prior art temporary pacemaker placement for patients with an underlying heart rhythm to sublimate the patient's venous flow to guide and direct the pacemaker lead into the right ventricle. The balloon is inflated when the distal end of the temporary pacemaker lead has been advanced through the introducer by the implant surgeon. For pressure based sensing that is independent of a dedicated pressure sensor, the balloon (bladder) remains inflated once the lead is positioned, then serves as a fluid (pneumatic or hydraulic) pressure sensor bulb, upon being connected to a fluid pressure transducer within the pacing controller. In the preferred embodiment of this invention, the connection to the pacing controller's fluid pressure transducer is accomplished via a quick-connect fluid (pneumatic or hydraulic) connector. Through this fluid pressure sensor bulb (inflatable bladder) and the interconnecting fluid medium, pressure events can be monitored via the external pacing controller to determine when the heart requires pacing.

Typically, the leads are connected to an external pacing controller for determination and configuration of pacing parameters.

Unlike traditional sensing, which is accomplished via the pacing leads, the pressure based sensing described above can be independently accomplished from anywhere within the fluid volume of the right ventricle, making it unnecessary to force a stiff pacing electrode tip into the myocardium in order to ensure quality sensing. Freed from the need to provide forceful contact with the myocardium for bioelectrical sensing, the distal pacing electrode can be designed solely for the more gentle contact required for the pacing function, thereby permitting a more bulbous tip, and significantly reducing the risk of myocardial perforation that exists, during implant, with either traditional or balloon guided, sharp radius (non-bulbous) tip designs.

### BRIEF DESCRIPTION OF THE DRAWINGS

For the purpose of illustrating the invention, there are shown in the accompanying drawings forms which are presently preferred; it being understood that the invention is not intended to be limited to the precise arrangements and instrumentalities shown.

FIG. 1 representatively depicts: an external pacing controller having integral data processing means; and a chamber of the heart, typically the right ventricle. A dedicated fluid pressure transducer is depicted within the chamber of the heart, as are two pacing electrodes. Within a catheter, electrical leads interconnect the transducer and each pacing electrode with the pacing controller.

FIG. 2 comprises a schematic diagram of the elements of FIG. 1, including the proximal leads exiting the catheter for the connection of the transducer and pacing leads to the pacing controller.

FIG. 3 representatively depicts: an external pacing controller having integral data processing means; and a chamber of the heart, typically the right ventricle. A dedicated fluid pressure sensor bulb is depicted within the chamber of the heart, as are two pacing electrodes. Within a catheter, an electrical lead interconnects each pacing electrode with the pacing controller, and an appropriate fluid-filled lead connects the fluid pressure sensor bulb with the pacing controller.

FIG. 4 comprises a schematic diagram of the elements of FIG. 3, including the proximal leads exiting the catheter for the connection of the sensor and pacing leads to the pacing controller.

FIG. 5 representatively depicts: an external pacing controller having integral data processing means; and a chamber of the heart, typically the right ventricle. A dedicated fluid pressure sensor bulb is depicted within the chamber of the heart, as are two pacing electrodes. Within a catheter, an electrical lead interconnects each pacing electrode with the pacing controller, and an appropriate fluid-filled lead connects the fluid pressure sensor bulb with the pacing controller. An inflatable bladder is located near the distal end of the catheter, and an access conduit to the inflatable bladder is included.

FIG. 6 comprises a schematic diagram of the elements of FIG. 5, including the proximal leads exiting the catheter for the connection of the sensor and pacing leads to the pacing controller and for fluid access to the inflatable bladder.

FIG. 7 representatively depicts: an external pacing controller having integral data processing means; and a chamber of the heart, typically the right ventricle. Two pacing electrodes are depicted within the chamber of the heart. Within a catheter, an electrical lead interconnects each pacing electrode with the pacing controller. An inflatable bladder is located near the distal end of the catheter, and a fluid-filled access conduit to the inflatable bladder is included.

FIG. 8 comprises a schematic diagram of the elements of FIG. 7, including the proximal leads exiting the catheter for the connection of the sensor and pacing leads to the pacing controller and for fluid-filled access to the inflatable bladder.

FIG. 9 representatively depicts: an external pacing controller having integral data processing means; and a chamber of the heart, typically the right ventricle. A dedicated fluid pressure transducer is depicted within the chamber of the heart, as are two pacing electrodes. Within a catheter, an electrical lead interconnects each pacing electrode and the fluid pressure transducer with the pacing controller. An inflatable bladder is located near the distal end of the catheter, and a fluid-filled access conduit to the inflatable bladder is included.

FIG. 10 comprises a schematic diagram of the elements of FIG. 5, including the proximal leads exiting the catheter for the connection of the transducer and pacing leads to the pacing controller and for fluid access to the inflatable bladder.

### LIST AND DESCRIPTIONS OF DRAWING ITEMS BY REFERENCE NUMBER

Item 100 depicts a patient's external pacing controller, incorporating integral data processing means and connection means for sense and pacing leads.

Item 102 is a simplified representation of a pacing catheter, incorporating pacing electrodes 110 and 112, pacing leads 210 and 212, fluid pressure transducer 108, and sense lead 208.

Item 106 represents a chamber of the heart, most typically the right ventricle.

Item 108 represents a dedicated fluid pressure transducer, positioned in heart chamber 106. The function of fluid pressure transducer 108 is to enable measurements by pacing controller 100 of intracardiac blood pressure variations for monitoring of the patient's cardiac pacing. Fluid pressure transducer 108 outputs on electrical sense lead 208 an electrical signal indicative of the fitted patient's intracardiac blood pressure variations.

Item 110 represents a conventional pacing electrode. Pacing electrode 110 is connected to pacing controller 100 via pacing lead 210.

Item 112 represents a blunted tip, otherwise conventional, pacing electrode. Pacing electrode 112 is connected to pacing controller 100 via pacing lead 212.

Item 208 represents a sense lead exiting pacing catheter 102 to interconnect the electrical signal output of fluid pressure transducer 108 with pacing controller 100. Sense lead 208 comprises an electrical lead, connected to an electrical terminal on pacing controller 100.

Item 210 represents an electrical lead exiting catheter 102 to interconnect pacing electrode 110 with pacing controller 100.

Item 212 represents an electrical lead exiting catheter 102 to interconnect pacing electrode 112 with pacing controller 100.

Item 300 depicts a patient's external pacing controller, incorporating integral data processing means and connection means for sense and pacing leads. Pacing controller 300 incorporates an (unclaimed) integral fluid pressure transducer. Pacing controller 300's fluid pressure transducer is fitted with an (unclaimed) miniature quick-connect fluid connector that mates with a connector on fluid pressure sense lead 408. Fluid pressure variations in fluid pressure sense lead 408, transmitted from fluid pressure sense bulb 308 to pacing controller 300's integral fluid pressure transducer, are indicative of the fitted patient's intracardiac blood pressure variations.

Item 302 is a simplified representation of a pacing catheter, incorporating pacing electrodes 310 and 312, pacing leads 410 and 412, fluid pressure sensor bulb 308, and fluid pressure sense lead 408.

Item 308 represents a dedicated fluid pressure sensor bulb, positioned in heart chamber 106. The function of fluid pressure sensor bulb 308 is to enable measurements by pacing controller 300 of intracardiac blood pressure variations for monitoring of the patient's cardiac pacing. Fluid pressure sensor bulb 308 transmits fluid pressure variations on fluid pressure sense lead 408. Said fluid pressure variations comprise a fluid pressure signal indicative of the fitted patient's intracardiac blood pressure variations. Fluid pressure sense lead 408 comprises a fluid (hydraulic or pneumatic) conduit, connected to a mating (unclaimed) quick-connect fluid connector on pacing controller 300.

Item 310 represents a conventional pacing electrode. Pacing electrode 310 is connected to pacing controller 300 via pacing lead 410.

Item 312 represents a blunted tip, otherwise conventional, pacing electrode. Pacing electrode 312 is connected to pacing controller 300 via pacing lead 412.

Item 408 represents a fluid-filled fluid (hydraulic or pneumatic) sense lead exiting pacing catheter 302 to interconnect the fluid signal output of fluid pressure sensor bulb 308 with pacing controller 300's integral fluid pressure transducer. The proximal end of fluid pressure sense lead 408 is fitted with a miniature (unclaimed) quick-connect fluid connector that mates with a connector on pacing controller 300's integral fluid pressure transducer.

Item 410 represents an electrical lead exiting catheter 302 to interconnect pacing electrode 310 with pacing controller 300.

Item 412 represents an electrical lead exiting catheter 302 to interconnect pacing electrode 312 with pacing controller 300.

Item 500 depicts a patient's external pacing controller, incorporating integral data processing means and connection means for sense and pacing leads. Pacing controller 500 incorporates an integral (unclaimed) fluid pressure transducer. Pacing controller 500's fluid pressure transducer is fitted with a miniature (unclaimed) quick-connect fluid connector that mates with a connector on sense lead 608. Fluid pressure variations in sense lead 608, transmitted from fluid pressure sense bulb 508 to pacing controller 500's integral fluid pressure transducer, are indicative of the fitted patient's intracardiac blood pressure variations.

Item 502 is a simplified representation of a pacing catheter, incorporating pacing electrodes 510 and 512, pacing leads 610 and 612, fluid pressure sensor bulb 508, fluid pressure sense lead 608, inflatable bladder 514, and bladder access conduit 614. In part, pacing catheter 502 resembles a catheter type commonly used in prior art temporary pacemaker placement for a patient with an underlying heart rhythm to sublimate the patient's venous flow to guide and direct pacing catheter 502 into patient's right ventricle 106.

Item 508 represents a dedicated fluid pressure sensor bulb, positioned in heart chamber 106. The function of fluid pressure sensor bulb 508 is to enable measurements by pacing controller 500 of intracardiac blood pressure variations for monitoring of the patient's cardiac pacing. Fluid pressure sensor bulb 508 transmits fluid pressure variations on fluid pressure sense lead 608. Said fluid pressure variations comprise a fluid pressure signal indicative of the fitted patient's intracardiac blood pressure variations. Fluid pressure sense lead 608 comprises a fluid (hydraulic or pneumatic) conduit, connected to a mating (unclaimed) quick-connect fluid connector on pacing controller 500.

Item 510 represents a conventional pacing electrode. Pacing electrode 510 is connected to pacing controller 500 via pacing lead 610.

Item 512 represents a blunted tip, otherwise conventional, pacing electrode. Pacing electrode 512 is connected to pacing controller 500 via pacing lead 612.

Item 514 depicts an inflatable bladder incorporated in pacing catheter 502. The function of inflatable bladder 514 is to sublimate the patient's venous flow to assist the implant surgeon in guiding and directing pacing catheter 502 into patient's right ventricle.

Item 608 represents a fluid-filled fluid (hydraulic or pneumatic) sense lead exiting pacing catheter 502 to interconnect the fluid signal output of fluid pressure sensor bulb 508 with pacing controller 500's integral fluid pressure transducer. The proximal end of fluid pressure sense lead 608 is fitted with a miniature (unclaimed) quick-connect fluid connector that mates with a connector on pacing controller 500's integral fluid pressure transducer.

Item 610 represents an electrical lead exiting catheter 502 to interconnect pacing electrode 510 with pacing controller 500.

Item 612 represents an electrical lead exiting catheter 502 to interconnect pacing electrode 512 with pacing controller 500.

Item 614 represents an access conduit exiting pacing catheter 502 for interconnecting inflatable bladder 514 with an (unclaimed) external device for controlling the inflation and deflation of inflatable bladder 514.

Item 700 depicts a patient's external pacing controller, incorporating integral data processing means and connection means for sense and pacing leads. Pacing controller 700 incorporates an integral (unclaimed) fluid pressure transducer. Pacing controller 700's fluid pressure transducer is fitted with a miniature (unclaimed) quick-connect fluid connector that mates with a connector on bladder access conduit 814. Fluid pressure variations in bladder access conduit 814, transmitted from inflatable bladder 714 to pacing controller 700's integral fluid pressure transducer, are indicative of the fitted patient's intracardiac blood pressure variations.

Item 702 is a simplified representation of a pacing catheter, incorporating pacing electrodes 710 and 712, pacing leads 810 and 812, inflatable bladder 714, and bladder access conduit 814. In part, pacing catheter 702 resembles a catheter type commonly used in prior art temporary pacemaker placement for a patient with an underlying heart rhythm to sublimate the patient's venous flow to guide and direct pacing catheter 702 into patient's right ventricle 106.

Item 710 represents a conventional pacing electrode. Pacing electrode 710 is connected to pacing controller 700 via pacing lead 810.

Item 712 represents a blunted tip, otherwise conventional, pacing electrode. Pacing electrode 712 is connected to pacing controller 700 via pacing lead 812.

Item 714 depicts an inflatable bladder incorporated in pacing catheter 702. Inflatable bladder 714 has two functions. Its initial function is to sublimate the patient's venous flow to assist the implant surgeon in guiding and directing pacing catheter 502 to patient's right ventricle 106. Its subsequent function is to act as an efficient and robust fluid pressure sensor bulb.

Item 810 represents an electrical lead exiting catheter 702 to interconnect pacing electrode 710 with pacing controller 700.

Item 812 represents an electrical lead exiting catheter 702 to interconnect pacing electrode 712 with pacing controller 700.

Item 814 represents a fluid-filled access conduit exiting pacing catheter 702 for interconnecting inflatable bladder 514 with pacing controller 700's fluid pressure transducer or to an (unclaimed) external device for controlling the inflation and deflation of inflatable bladder 714. The proximal end of access conduit 814 is fitted with a miniature (unclaimed) quick-connect fluid connector that mates with that on pacing controller 700's fluid pressure transducer and with that on any external inflation inflation/deflation control device employed.

Item 900 depicts a patient's external pacing controller, incorporating integral data processing means and connection means for sense and pacing leads.

Item 902 is a simplified representation of a pacing catheter, incorporating pacing electrodes 910 and 912, pacing leads 1010 and 1012, fluid pressure transducer 908, and sense lead 1008.

Item 908 represents a dedicated fluid pressure transducer, positioned in heart chamber 106. The function of fluid pressure transducer 908 is to enable measurements by pacing controller 900 of intracardiac blood pressure variations for monitoring of the patient's cardiac pacing. Fluid pressure transducer 908 outputs on electrical sense lead 1008 an electrical signal indicative of the fitted patient's intracardiac blood pressure variations.

Item 910 represents a conventional pacing electrode. Pacing electrode 910 is connected to pacing controller 900 via pacing lead 1010.

Item 912 represents a blunted tip, otherwise conventional, pacing electrode. Pacing electrode 912 is connected to pacing controller 900 via pacing lead 1012.

Item 914 depicts an inflatable bladder incorporated in pacing catheter 902. The function of inflatable bladder 914 is to sublimate the patient's venous flow to assist the implant surgeon in guiding and directing pacing catheter 902 into patient's right ventricle.

Item 1008 represents a sense lead exiting pacing catheter 902 to interconnect the electrical signal output of fluid pressure transducer 908 with pacing controller 900. Sense lead 1008 comprises an electrical lead, connected to an electrical terminal on pacing controller 900.

Item 1010 represents an electrical lead exiting catheter 902 to interconnect pacing electrode 910 with pacing controller 900.

Item 1012 represents an electrical lead exiting catheter 902 to interconnect pacing electrode 912 with pacing controller 900.

Item 1014 represents an access conduit exiting pacing catheter 902 for interconnecting inflatable bladder 914 with an (unclaimed) external device for controlling the inflation and deflation of inflatable bladder 914.

### Detailed Description of the Preferred Embodiments

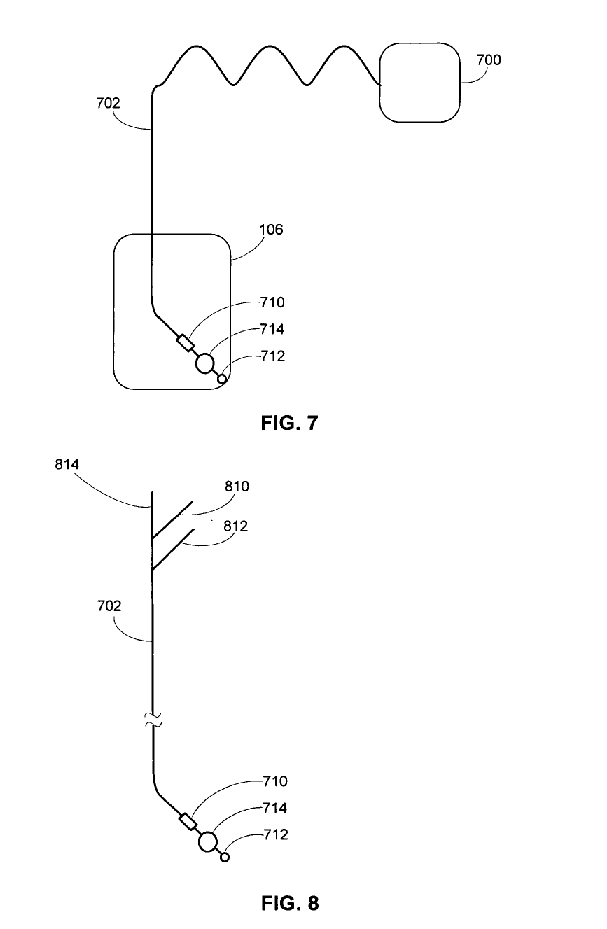

Refer to FIG. 7 and to FIG. 8. Inflatable bladder 714 serves dual purposes. It forms a part of balloon (bladder) guided pacing catheter 702, in part resembling a catheter type commonly used in prior art temporary pacemaker placement for a patient with an underlying heart rhythm to sublimate the patient's venous flow to assist the implant surgeon in guiding and directing pacing catheter 702 into patient's right ventricle 106 such that inflatable bladder 714 and pacing electrodes 710 and 712 are positioned within patient's right ventricle 106. Pacing electrodes 710 and 712 are then connected via pacing leads 810 and 812 to pacing controller 700, and fluid pressure sensor bulb (inflatable bladder) 714 is connected via a fluid column within bladder access conduit 814 to an (unclaimed) fluid pressure transducer integral within pacing controller 700.

To accomplish its placement assist function, balloon (bladder) 714 is inflated when the distal end of temporary pacing catheter 702 has been advanced through a conventional introducer by the implant surgeon.

For pressure based sensing that is independent of a dedicated pressure sensor such as fluid pressure sensor bulb 508, balloon (bladder) 714 remains inflated once the lead is positioned. Balloon (bladder) 714 then serves as a fluid (pneumatic or hydraulic) pressure sensor bulb, upon being connected via fluid-filled bladder access conduit 814 to an (unclaimed) fluid pressure transducer integral to pacing controller 700. The connection to pacing controller 700's fluid pressure transducer is accomplished via an (unclaimed) quick-connect fluid (pneumatic or hydraulic) connector.

By monitoring variations in fluid pressure sensor bulb (inflatable bladder) 714, pressure events can be monitored by external pacing controller 700 to determine when the heart requires pacing. Intracardiac blood pressure variations within cardiac chamber 106 cause corresponding variations in fluid pressure sensor bulb (bladder) 714. Variations within fluid pressure sensor bulb 714 are transmitted up fluid-filled bladder access conduit 814 to controller 700's fluid pressure transducer, and converted to electrical signals that can be analyzed by pacing controller 700 to establish pacing parameters customized to the fitted patent's needs. Such pacing parameters are used to configure the operation of external pacing controller 700 to assist the patient's cardiac function.

Pressure based sensing does not require electrode contact with the myocardium. Electrode contact with the myocardium serves only the pacing function, which does not require forceful myocardial impingement by pacing electrode 712. Consequently, the blunted tip of pacing electrode 712 adequately fulfills its function, and significantly decreases the probability of myocardial perforation.

### Detailed Description of a Second Embodiment of the Invention

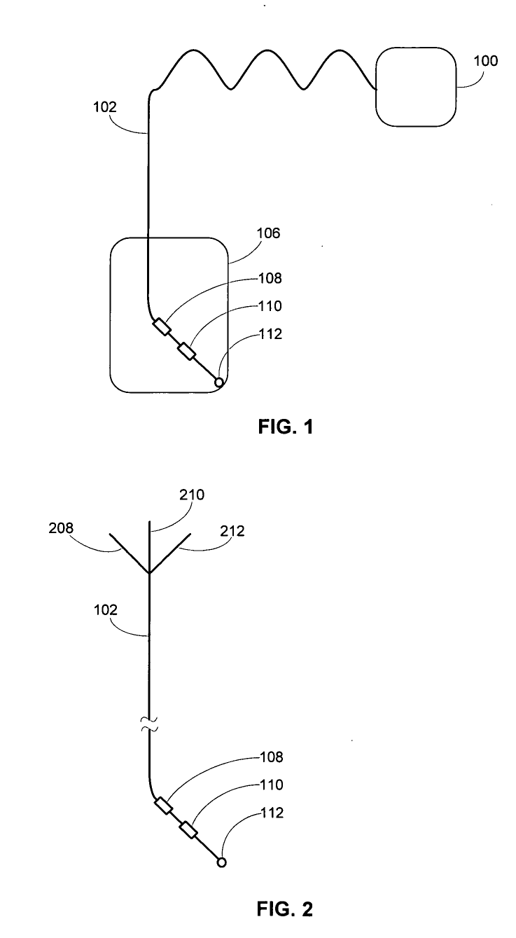

Refer to FIG. 1 and to FIG. 2. Temporary pacing catheter 102 is advanced through a conventional introducer by the implant surgeon such that fluid pressure transducer 108 and pacing electrodes 110 and 112 are positioned within patient's right ventricle 106. Pacing electrodes 110 and 112 are then connected via pacing leads 210 and 212 to pacing controller 100, and fluid pressure transducer 108 is connected via electrical sense lead 208 to pacing controller 100.

Variations in intracardiac blood pressure are transmitted as electrical signals from fluid pressure transducer 108, transmitted via sense lead 208 to controller 100, where they are analyzed by pacing controller 100 to establish pacing parameters customized to the fitted patent's needs. Such pacing parameters are used to configure the operation of external pacing controller 100 to assist the patient's cardiac function.

By monitoring intracardiac blood pressure variations transmitted from fluid pressure transducer 108 via sense lead 208 to pacing controller 100, pressure events can be monitored by external pacing controller 100 to determine when the heart requires pacing. Such pressure events can be analyzed by pacing controller 100 to establish pacing parameters customized to the fitted patent's needs to assist the patient's cardiac function.

Pressure based sensing does not require electrode contact with the myocardium. Electrode contact with the myocardium serves only the pacing function, which does not require forceful myocardial impingement by pacing electrode 112. Consequently, the blunted tip of pacing electrode 112 adequately fulfills its function, and significantly decreases the probability of myocardial perforation.

### Detailed Description of a Third Embodiment of the Invention

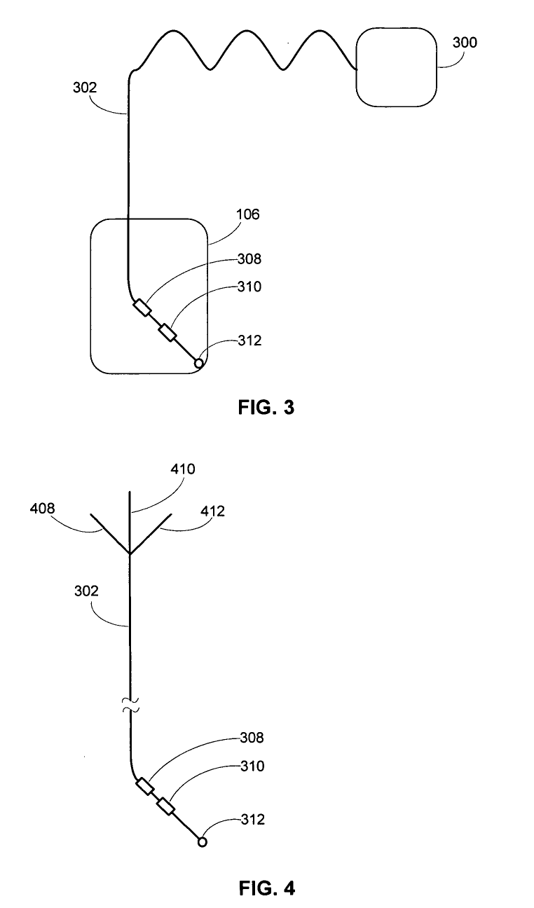

Refer to FIG. 3 and to FIG. 4. Temporary pacing catheter 302 is advanced through a conventional introducer by the implant surgeon such that fluid pressure sensor bulb 308 and pacing electrodes 310 and 312 are positioned within patient's right ventricle 106. Pacing electrodes 310 and 312 are then connected via pacing leads 410 and 412 to pacing controller 300, and fluid pressure sensor bulb 308 is connected via fluid-filled (hydraulic or pneumatic) sense lead 408 to an (unclaimed) fluid pressure transducer integral within pacing controller 300. The connection to pacing controller 300's fluid pressure transducer is accomplished via an (unclaimed) quick-connect fluid (pneumatic or hydraulic) connector.

Variations in intracardiac blood pressure are transmitted from remote fluid pressure sensor bulb 308, and transmitted up fluid-filled fluid sense lead 408 to controller 300's fluid pressure transducer, where they are converted to electrical signals that can be analyzed by pacing controller 300 to establish pacing parameters customized to the fitted patent's needs. Such pacing parameters are used to configure the operation of external pacing controller 300 to assist the patient's cardiac function.

By monitoring intracardiac blood pressure variations transmitted from remote fluid pressure sensor bulb 308 via fluid-filled fluid sense lead 408 to pacing controller 300's integral fluid pressure transducer where they are converted to electrical signals, pressure events can be monitored and analyzed by external pacing controller 300 to determine when the heart requires pacing. Such pressure events, when converted to electrical signals, can be analyzed by pacing controller 300 to establish pacing parameters customized to the fitted patent's needs. Such pacing parameters are used to configure the operation of external pacing controller 300 to assist the patient's cardiac function.

Pressure based sensing does not require electrode contact with the myocardium. Electrode contact with the myocardium serves only the pacing function, which does not require forceful, myocardial impingement by pacing electrode 312. Consequently, the blunted tip of pacing electrode 312 adequately fulfills its function, and significantly decreases the probability of myocardial perforation.

### Detailed Description of a Fourth Embodiment of the Invention

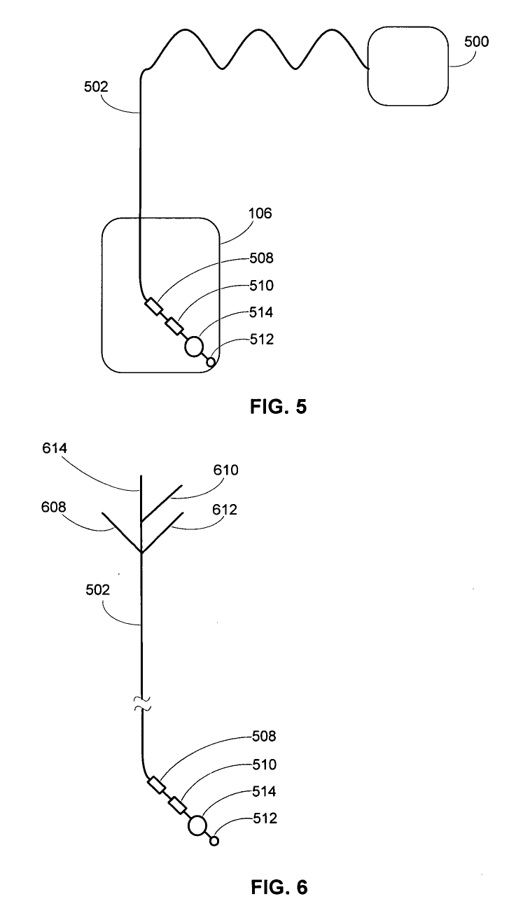

Refer to FIG. 5 and to FIG. 6. Inflatable bladder 514 forms a part of balloon (bladder) guided pacing catheter 502, resembling a type commonly used in prior art temporary pacemaker placement for a patient with an underlying heart rhythm to sublimate the patient's venous flow to assist the implant surgeon in guiding and directing pacing catheter 502 into patient's right ventricle 106 such that fluid pressure sensor bulb 508 and pacing electrodes 510 and 512 are positioned within patient's right ventricle 106. Pacing electrodes 510 and 512 are then connected via pacing leads 610 and 612 to pacing controller 500, and fluid pressure sensor bulb 508 is connected via fluid (hydraulic or pneumatic) sense lead 608 to an (unclaimed) fluid pressure transducer integral within pacing controller 500. The connection to pacing controller 500's fluid pressure transducer is accomplished via an (unclaimed) quick-connect fluid (pneumatic or hydraulic) connector.

To accomplish its placement assist function, inflatable balloon (bladder) 514 is inflated when the distal end of temporary pacing catheter 502 has been advanced through a conventional introducer by the implant surgeon. Inflatable bladder 514 is typically deflated once fluid pressure sensor bulb 508 and pacing electrodes 510 and 512 are positioned within patient's right ventricle 106.

Variations in intracardiac blood pressure are transmitted from remote fluid pressure sensor bulb 508, and transmitted up fluid sense lead 608 to controller 500's fluid pressure transducer, where they are converted to electrical signals and analyzed by pacing controller 500 to establish pacing parameters customized to the fitted patent's needs. Such pacing parameters are used to configure the operation of external pacing controller 500 to assist the patient's cardiac function.

Pressure based sensing does not require electrode contact with the myocardium. Electrode contact with the myocardium serves only the pacing function, which does not require forceful myocardial impingement by pacing electrode 512. Consequently, the blunted tip of pacing electrode 512 adequately fulfills its function, and significantly decreases the probability of myocardial perforation.

### Detailed Description of a Fifth Embodiment of the Invention

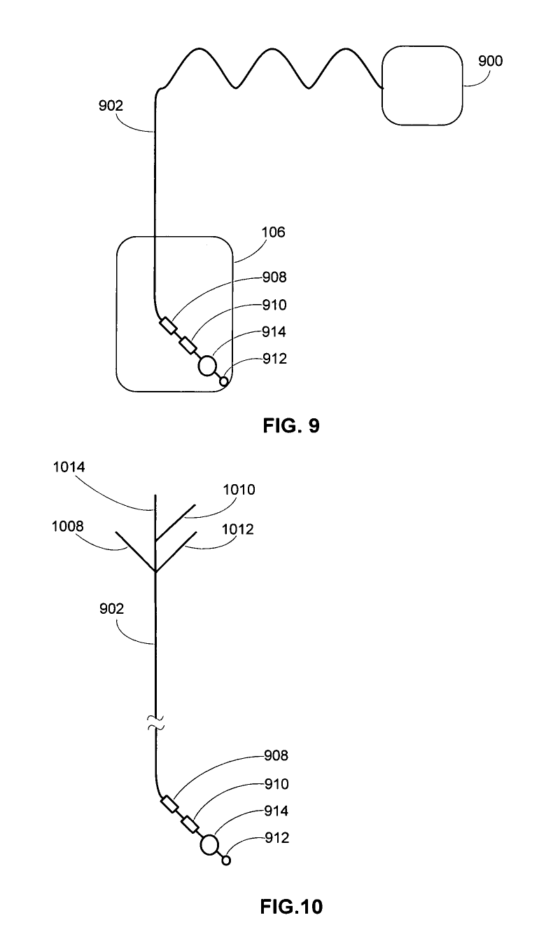

Refer to FIG. 9 and to FIG. 10. Inflatable bladder 914 forms a part of bladder (balloon) guided pacing catheter 902, in part resembling a catheter type commonly used in prior art temporary pacemaker placement for a patient with an underlying heart rhythm to sublimate the patient's venous flow to assist the implant surgeon in guiding and directing pacing catheter 902 into patient's right ventricle 106 such that fluid pressure transducer 908 and pacing electrodes 910 and 912 are positioned within patient's right ventricle 106. Pacing electrodes 910 and 912 are then connected via pacing leads 1010 and 1012 to pacing controller 900, and fluid pressure transducer 908 is connected to pacing controller 900 via sense lead 1008.

To accomplish its placement assist function, inflatable balloon (bladder) 914 is inflated when the distal end of temporary pacing catheter 902 has been advanced through a conventional introducer by the implant surgeon. Inflatable bladder 914 is typically deflated once fluid pressure transducer 908 and pacing electrodes 910 and 912 are positioned within patient's right ventricle 106.

Variations in intracardiac blood pressure are transmitted as electrical signals from fluid pressure transducer 908, transmitted via sense lead 1008 to controller 900, where they are analyzed by pacing controller 900 to establish pacing parameters customized to the fitted patent's needs. Such pacing parameters are used to configure the operation of external pacing controller 900 to assist the patient's cardiac function. By monitoring intracardiac blood pressure variations transmitted from fluid pressure transducer 908 via sense lead 1008 to pacing controller 900, pressure events can be monitored by external pacing controller 900 to determine when the heart requires pacing. Such pressure events can be analyzed by pacing controller 900 to establish pacing parameters customized to the fitted patent's needs to assist the patient's cardiac function.

Pressure based sensing does not require electrode contact with the myocardium. Electrode contact with the myocardium serves only the pacing function, which does not require forceful myocardial impingement by pacing electrode 912. Consequently, the blunted tip of pacing electrode 912 adequately fulfills its function, and significantly decreases the probability of myocardial perforation.

### Other Embodiments and Applications of this Invention

Essentially, the preferred embodiment of this invention has been described along with several additional feasible embodiments. Still other embodiments, applications, and ramifications are possible within the scope of this invention. Thus, the scope of the invention should be determined by the appended claims and their legal equivalents rather than by the examples given.

For example, the pacing catheter, itself, can be used as the bladder access conduit.

As another example, only fluid pressure variation has been mentioned as the non-bioelectrical monitoring parameter to yield a non-bioelectrical cardiogram of cardiac function; however, volumetric fluid pressure soundings of cardiac chamber dimensions can also be employed as the required non-bioelectrical monitoring parameter.

As yet another example, the non-bioelectrical monitoring sensor does not have to be in same cardiac chamber as the pacing electrodes.

### Glossary

The terms “blunted tip” and “more bulbous tip”, as used herein, are intended to mean a less stiff distal pacing lead tip that is of larger radius than a conventional tip.

The terms “chamber of the heart”, “heart chamber”, and “cardiac chamber”, as used herein, are intended to refer to any chamber of the heart.

The term “configurable by the surgeon”, as used herein, is intended to refer to features of the pacemaker/controller that are configured by the surgeon at the time of implantation, and/or which may subsequently be reconfigured via conventional external controller.

The word “conventional”, as used herein, is intended to refer to means and/or methods not unique to this invention.

The word “external”, as used herein, is intended to refer to something that is external to the patient's body.

The term “lead set”, as used herein, is intended to refer to the leads integral to a pacing catheter.

The term “non-bioelectrical”, as used herein, is intended to mean not directly monitoring biological electrical signals.

The word “unclaimed”, as applied to an item herein, is intended to mean the design of the item is not claimed as a part of this invention.

The present invention may be embodied in other specific forms without departing from the spirit or essential attributes thereof and accordingly reference should be made to the appended claims rather than to the foregoing specification as indicating the scope of the invention.

We claim:

1. A pacing catheter for use with a temporary cardiac pacemaker comprising:
said catheter including a distal end and a proximal end;
a lead set carried by the distal end of said catheter and being adapted to be positioned in a chamber of the heart of a patient;
said proximal end of said catheter being adapted to be connected to a controller located external to said patient;
said lead set including electrodes adapted to be connected to said controller for providing an electrical charge to said heart for pacing the same;
fluid pressure sensing means carried by said distal end of said catheter adjacent said electrodes, and
means connecting said fluid pressure sensing means to said controller for communicating changes in the fluid pressure sensed by said fluid pressure sensing means to said controller.

2. The pacing catheter as claimed in claim 1 wherein said fluid pressure sensing means is a fluid pressure transducer.

3. The pacing catheter as claimed in claim 1 wherein said fluid pressure sensing means is a fluid pressure transducer and wherein means are provided for electrically connecting said transducer to said controller.

4. The pacing catheter as claimed in claim 1 wherein said fluid pressure sensing means is a fluid pressure sensing bulb and wherein said catheter includes a fluid pressure conduit for conveying the sensed fluid pressure to said controller.

5. The pacing catheter as claimed in claim 1 wherein said catheter includes an inflatable balloon adjacent the distal end thereof for guiding the catheter into said chamber of the heart.

6. The pacing catheter as claimed in claim 5 wherein said inflatable balloon also functions as said fluid pressure sensing mean.

7. The pacing catheter as claimed in claim 1 wherein said distal end of said catheter includes a blunted tip.
